(19) United States
(12) Patent Application Publication
(10) Pub. No.: US 2008/0177189 A1
(43) Pub. Date: Jul. 24, 2008

(54) Photoplethysmography sensor

(75) Inventors: Youn Ho Kim, Hwaseong-si (KR); Kun-Kook Park, Suwon-si (KR); Kun Soo Shin, Seongnam-si (KR); Sang Kon Bae, Seongnam-si (KR); Soo Kwan Kim, Seongnam-si (KR)

Correspondence Address: STAAS & HALSEY LLP, SUITE 700, 1201 NEW YORK AVENUE, N.W., WASHINGTON, DC, 20005, US

(73) Assignee: SAMSUNG ELECTRONICS CO., LTD., Suwon-si (KR)

(21) Appl. No.: 11/984,822

(22) Filed: Nov. 21, 2007

(30) Foreign Application Priority Data
Jan. 19, 2007 (KR) 10-2007-0006296

(51) Int. Cl. A61B 5/02

(52) U.S. Cl. 600/504

(57) ABSTRACT

A photoplethysmography (PPG) sensor includes: a conductive member contacting with the skin of a user; a luminous element disposed on the conductive member to emit light towards the skin of the user through the conductive member; and a photo detector detecting the light emitted towards the skin of the user.

### CROSS-REFERENCE TO RELATED APPLICATIONS

This application claims the benefit of Korean Patent Application No. 10-2007-0006296, filed on Jan. 19, 2007, in the Korean Intellectual Property Office, the disclosure of which is incorporated herein by reference.

### BACKGROUND

1. Field

The present invention relates to a photoplethysmography (PPG) sensor, and more particularly, to a PPG sensor which provides a conductive member, such as indium-tin oxide (ITO) glass, and the like, on one surface of a sensor contacting with the skin of a user to induce a stable ground of the sensor, and thereby reduces noise even with respect to a weak PPG signal detected from an earlobe, and also more accurately and stably detects a PPG signal.

2. Description of the Related Art

As used in the present specification, the term “Ubiquitous” means an information communication environment where a user is free to access networks at any place without being conscious of the surrounding networks or computers. If Ubiquitous is commercialized, anyone can readily use information technology not only at home or in a car, but also even on a mountaintop. Also, the commercialization of Ubiquitous may expand the information technology industry or the scope corresponding thereto by increasing the number of computer users who are connected to networks. Because of its advantage that users can access networks without restriction to time and place, not to mention its portability and convenience, countries worldwide are expanding development and competing in Ubiquitous-related technology now.

Ubiquitous-related technology may be applied to a myriad of fields in human life. In particular, Ubiquitous-HealthCare (hereinafter, U-HealthCare) has recently been in the spotlight as a notable technology area due to the “well-being” boom. U-HealthCare means Ubiquitous technology which enables anyone to readily receive medical services at any time and at any place by installing medical service-related chips or sensors in places of the user's living space. With U-HealthCare, various types of medical attention, such as physical examinations, disease management, emergency care, consultation with a doctor and the like, which currently are only performed in hospitals, may be naturally integrated into our daily lives, thus may be accomplished without going to a hospital.

For example, a diabetic may wear a belt having a blood-sugar management system for blood-sugar management. A blood-sugar sensor attached to the belt may check the blood-sugar of the diabetic upon a specified occasion, and calculate the amount of required insulin corresponding thereto. When the blood-sugar of the diabetic becomes drastically low or high, the belt may provide the blood-sugar information to his/her attending physician via a wireless network, and the attending physician who has received the blood-sugar information may write out an optimal prescription or take the optimal action for the medical emergency.

As an example of U-HealthCare, a portable biosignal measurement device for measuring the user's biosignal using an optical sensor is being widely utilized. The user may carry the portable biosignal measurement device at all times and measure various types of biosignals, and thereby may be prepared for an emergency situation. Accordingly, the portable biosignal measurement device may be regarded as a device capable of showing advantages of U-HealthCare.

The portable biosignal measurement device includes a photoplethysmography (PPG) measurement device. A PPG includes information about a level of peripheral vasoconstriction, and an increase and decrease in a cardiac output. Therefore, a physiological status associated with an arterial tube may be understood using the PPG measurement device. Also, the PPG measurement device may be generally utilized as an auxiliary diagnostic device for a particular disease.

Generally, a PPG signal may be measured from a user's finger, earlobe, and the like. Specifically, a detector may detect the user's PPG signal by detecting light, passing through the finger, earlobe, and the like, from a light source. However, when a PPG signal is weak, for example, a PPG signal detected from the earlobe, and the like, a normal PPG signal may not be detected.

When a measurement device measures a PPG signal from a body portion corresponding to a weak signal source, such as the earlobe, and the like, a level of the PPG signal may be less than noise of the measurement device. Specifically, the level of the PPG signal may be less than a system noise level. Therefore, although the weak PPG signal is amplified, the system noise is also amplified, and thus a desired PPG signal may not be accurately detected.

The system noise may be affected by external environments, however, and generally may result from an unstable ground. Specifically, in the case of a portable PPG measurement device, a battery functions as the ground when measuring a PPG signal. In this instance, a standard for a stable ground is to set to the earth. When the battery functions as the ground as described above, a level of the battery is set to a value greater than a value when the earth functions as the ground, and thus a ground status becomes unstable. Accordingly, when the battery functions as the ground, the battery may not stably function as the ground like the earth ground, and thus the system noise may be set to a greater value. Specifically, when measuring a PPG signal from the earlobe, and the like, corresponding to a weak signal source, an accurate PPG signal may not be stably detected.

Accordingly, there is a need for a portable PPG measurement device capable of reducing system noise in a measurement device even when a signal strength is significantly weak, and thereby accurately and stably detecting a PPG signal.

### SUMMARY

In an aspect of the present invention, a photoplethysmography (PPG) sensor includes a conductive member, such as indium-tin oxide (ITO) glass, and the like, on one surface of an optical sensor contacting with the skin of a user, and thereby induces a more stable ground through the contacted skin of the user.

In another aspect of the present invention, a PPG sensor reduces system noise of a measurement device through a stable ground using the skin of a user, instead of utilizing a battery ground, and thereby more accurately and stably detects a PPG signal even from the earlobe, and the like, corresponding to a weak signal source.

According to an aspect of the present invention, a PPG sensor includes: a conductive member contacting with the skin of a user; a luminous element on the conductive member to emit light towards the skin of the user through the conductive member; and a photo detector detecting the light emitted towards the skin of the user.

According to another aspect of the present invention, a PPG sensor includes: a conductive member contacting with the skin of a user; a luminous element emitting light towards the skin of the user; and a photo detector on the conductive member to detect the light emitted towards the skin of the user through the conductive member.

According to still another aspect of the present invention, a PPG sensor includes: a conductive member contacting with the skin of a user; a luminous element on the conductive member to emit light towards the skin of the user through the conductive member; and a photo detector on the conductive member to detect the light emitted towards the skin of the user through the conductive member.

According to yet another aspect of the present invention, a PPG sensor includes: a luminous element emitting light towards the skin of a user; a first conductive member on one surface of the luminous element to contact with the skin of the user; a photo detector detecting the light emitted towards the skin of the user; and a second conductive member on one surface of the photo detector to contact with the skin of the user.

Additional aspects, features, and/or advantages of the invention will be set forth in part in the description which follows and, in part, will be apparent from the description, or may be learned by practice of the invention.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 illustrates a structure of a reflective PPG sensor according to an embodiment of the present invention;

FIG. 2 illustrates a structure of a transmitting PPG sensor according to an embodiment of the present invention;

FIG. 3 illustrates a structure of a reflective PPG sensor according to another embodiment of the present invention;

FIG. 4 illustrates a structure of a transmitting PPG sensor according to another embodiment of the present invention;

FIG. 5 illustrates a structure of a reflective PPG sensor according to still another embodiment of the present invention; and

FIG. 6 illustrates a structure of a transmitting PPG sensor according to still another embodiment of the present invention.

### DETAILED DESCRIPTION OF THE EMBODIMENTS

Reference will now be made in detail to exemplary embodiments of the present invention, examples of which are illustrated in the accompanying drawings, wherein like reference numerals refer to the like elements throughout. Embodiments are described below to explain the present invention by referring to the figures.

A photoplethysmography (PPG) sensor according to the present invention may be embodied as a partial configuration of a portable biosignal measurement device. The power of the portable biosignal measurement device may be embodied as a battery. Specifically, the PPG sensor according to the present invention is included in the portable biosignal measurement device. When measuring a PPG signal of a user, the battery may not function as a ground, and the body of the user may function as the ground.

Also, a sensor unit of the portable biosignal measurement device may be embodied into a form of an accessory, such as a headset, a ring, and the like. Specifically, although a user does not take a particular action, the user may measure the user's PPG signal via the PPG sensor according to the present invention while listening to music via a headset, and the like.

Hereinafter, embodiments of the present invention will be described with reference to the accompanying drawings.

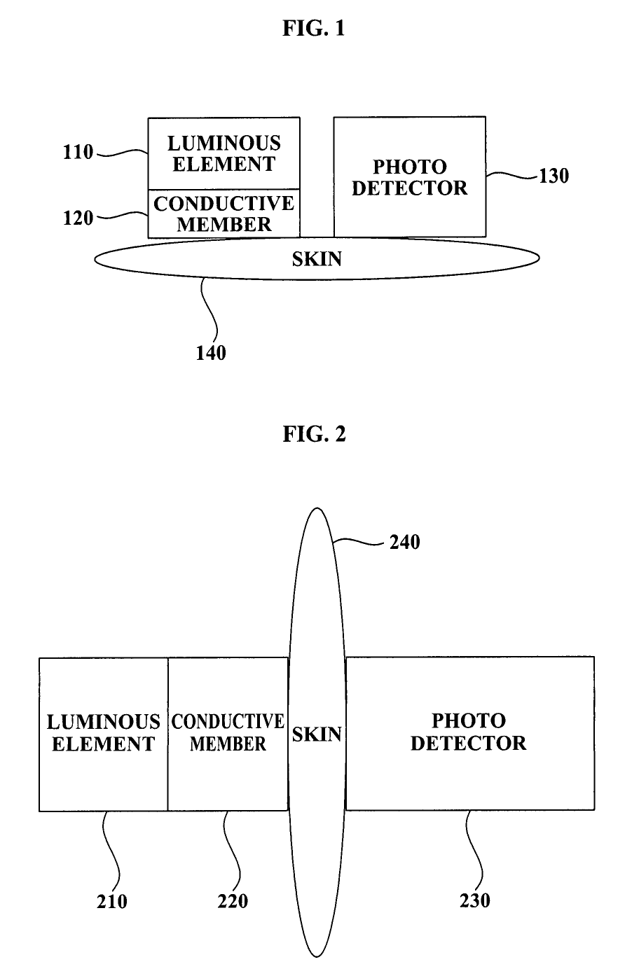

FIG. 1 illustrates a structure of a reflective PPG sensor according to an embodiment of the present invention.

The reflective PPG sensor according to the present embodiment may include a luminous element 110 and a photo detector 130 arranged in parallel on one surface of the skin of a user 140.

Also, the reflective PPG sensor may include a conductive member 120 on the luminous element 110. Specifically, one surface of the conductive member 120 may contact with the skin of the user 140, and the other surface of the conductive member 120, not contacting with the skin of the user 140, may be provided with the luminous element 110.

The conductive member 120 may include indium-tin oxide (ITO) glass. In this instance, the ITO glass may readily transmit light and may also include conductivity. Accordingly, the ITO glass may enable the light emitted from the luminous element 110 to be sufficiently transmitted towards the skin of the user 140.

Also, the conductive member 120 contacts with the skin of the user 140, and thereby makes the body of the user function as a ground. In this instance, the body of the user generally contacts with the earth, and thus, results in the earth functioning as the ground due to the conductive member 120.

As described above, when a PPG signal of a user is measured via a PPG sensor according to the present invention, the body of the user contacts the earth so that the earth functions as a ground, instead of utilizing a ground consisting of a battery of a portable biosignal measurement device including the PPG sensor. Accordingly, a more stable earth ground may be provided, instead of a battery ground, which is set to a comparatively greater value. Also, a PPG signal may be more accurately and stably detected, even from the earlobe, and the like, in which the PPG signal is generally weak.

The photo detector 130 detects the light from the skin of the user 140. Specifically, the photo detector 130 detects the light which is emitted towards the skin of the user 140 through the conductive member 120, and thereby is scattered or reflected from the skin of the user 140.

In this instance, the conductive member 120 may include ITO glass as described above. Also, the conductive member 120 may be formed of various types of materials which can readily transmit the light, and may also include conductivity.

The luminous element 110 may include a light emitting diode (LED). Also, in addition to the LED, to measure a PPG signal, the luminous element 110 may include any type of material which is widely utilized in the art to emit the light towards the skin of the user 140.

FIG. 2 illustrates a structure of a transmitting PPG sensor according to an embodiment of the present invention.

The transmitting PPG sensor according to the present embodiment may include a luminous element 210 on one surface of the skin of a user 240 and a photo detector 230 on the other surface of the skin of the user 240. Specifically, the transmitting PPG sensor may be formed in a type of an earring which is attachable onto the earlobe of the user.

Also, the transmitting PPG sensor may include a conductive member 220 on the luminous element 210. Specifically, one surface of the conductive member 220 may contact with the skin of the user 240, and the other surface of the conductive member 220, not contacting with the skin of the user 240, may be provided with the luminous element 210.

The conductive member 220 may include ITO glass. In this instance, the ITO glass may readily transmit light, and may also include conductivity. Accordingly, the ITO glass may enable the light emitted from the luminous element 210 to be sufficiently transmitted towards the skin of the user 240.

Also, the conductive member 220 contacts with the skin of the user 240, and thereby makes the body of the user function as a ground. In this instance, the body of the user generally contacts with the earth, and thus, results in the earth functioning as the ground due to the conductive member 220.

As described above, when a PPG signal of a user is measured via a PPG sensor according to the present invention, the body of the user connects with the earth, so that the earth functions as a ground, instead of utilizing a ground consisting of a battery of a portable biosignal measurement device including the PPG sensor. Accordingly, a more stable earth ground may be provided, instead of a battery ground, which is set to a comparatively greater value. Also, a PPG signal may be more accurately and stably detected even from the earlobe, and the like, in which the PPG signal is generally weak.

The photo detector 230 detects the light from the skin of the user 240. Specifically, the photo detector 230 detects the light which is emitted towards the skin of the user 240 through the conductive member 220, and thereby is scattered or reflected from the skin of the user 240.

In this instance, the conductive member 220 may include ITO glass as described above. Also, the conductive member 220 may be formed of various types of materials which can readily transmit the light, and may also include conductivity.

The luminous element 210 may include an LED. Also, in addition to the LED, to measure a PPG signal, the luminous element 210 may include any type of material which is widely utilized in the art to emit the light towards the skin of the user 240.

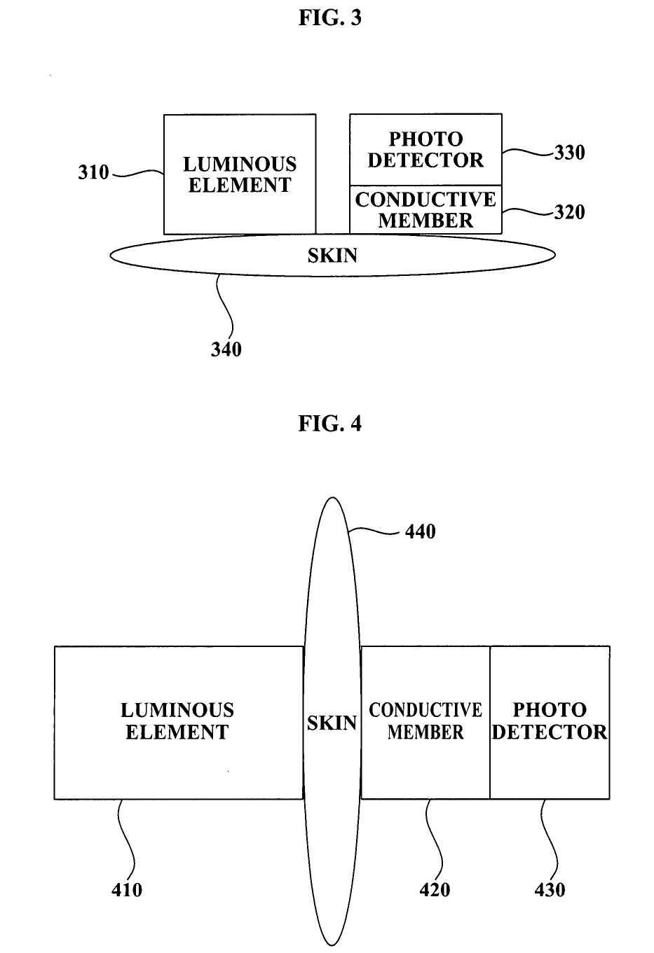

FIG. 3 illustrates a structure of a reflective PPG sensor according to another embodiment of the present invention.

The reflective PPG sensor according to the present embodiment may include a luminous element 310 and a photo detector 330 arranged in parallel on one surface of the skin of a user 340.

Also, the reflective PPG sensor may include a conductive member 320 on the photo detector 330. Specifically, one surface of the conductive member 320 may contact with the skin of the user 340, and the other surface of the conductive member 320, not contacting with the skin of the user 340, may be provided with the photo detector 330.

The conductive member 320 may include ITO glass. In this instance, the ITO glass may readily transmit light and may also include, conductivity. Accordingly, the ITO glass may enable the photo detector 330 to sufficiently detect the light emitted from the luminous element 310 towards the skin of the user 340.

Also, the conductive member 320 contacts with the skin of the user 340, and thereby makes the body of the user function as a ground. In this instance, the body of the user generally contacts with the earth, and thus, results in the earth functioning as the ground due to the conductive member 320.

As described above, when a PPG signal of a user is measured via a PPG sensor according to the present invention, the body of the user generally connects with the earth so that the earth functions as a ground, instead of utilizing a ground consisting of a battery of a portable biosignal measurement device including the PPG sensor. Accordingly, a more stable earth ground may be provided, instead of a battery ground which is set to a comparatively greater value. Also, a PPG signal may be more accurately and stably detected even from the earlobe, and the like, in which the PPG signal is generally weak.

The photo detector 330 detects the light from the skin of the user 340. Specifically, the photo detector 330 detects the light which is emitted from the luminous element 310 towards the skin of the user 340, and thereby is scattered or reflected through the conductive member 320.

In this instance, the conductive member 320 may include ITO glass as described above. Also, the conductive member 320 may be formed of various types of materials which can readily transmit the light, and may also include conductivity.

The luminous element 310 may include an LED. Also, in addition to the LED, to measure a PPG signal, the luminous element 310 may include any type of material which is widely utilized in the art to emit the light towards the skin of the user 340.

FIG. 4 illustrates a structure of a transmitting PPG sensor according to another embodiment of the present invention.

The transmitting PPG sensor according to the present embodiment may include a luminous element 410 on one surface of the skin of a user 440 and a photo detector 430 on the other surface of the skin of the user 440. Specifically, the transmitting PPG sensor may be formed in a type of an earring which is attachable onto the earlobe of the user.

Also, the transmitting PPG sensor may include a conductive member 420 on the photo detector 430. Specifically, one surface of the conductive member 420 may contact with the skin of the user 440, and the other surface of the conductive member 420, not contacting with the skin of the user 440, may be provided with the photo detector 430.

The conductive member 420 may include ITO glass. In this instance, the ITO glass may readily transmit light, and may also include conductivity. Accordingly, the ITO glass may enable the photo detector 430 to sufficiently detect the light emitted from the luminous element 410 towards the skin of the user 440.

Also, the conductive member 420 contacts with the skin of the user 440, and thereby makes the body of the user function as a ground. In this instance, the body of the user generally contacts with the earth, and thus results in the earth functioning as the ground due to the conductive member 420.

As described above, when a PPG signal of a user is measured via a PPG sensor according to the present invention, the body of the user contacts the earth so that the earth, functions as a ground, instead of utilizing a ground consisting of a battery of a portable biosignal measurement device including the PPG sensor. Accordingly, a more stable earth ground may be provided, instead of a battery ground, which is set to a comparatively greater value. Also, a PPG signal may be more accurately and stably detected even from the earlobe, and the like, in which the PPG signal is generally weak.

The photo detector 430 detects the light from the skin of the user 440. Specifically, the photo detector 430 detects the light, which is emitted from the luminous element 410 towards the skin of the user 440, and thereby is scattered or reflected from the skin of the user 440 through the conductive member 420.

In this instance, the conductive member 420 may include ITO glass as described above. Also, the conductive member 420 may be formed of various types of materials which can readily transmit the light and may also include conductivity.

The luminous element 410 may include an LED. Also, in addition to the LED, to measure a PPG signal, the luminous element 410 may include any type of material which is widely utilized in the art to emit the light towards the skin of the user 440.

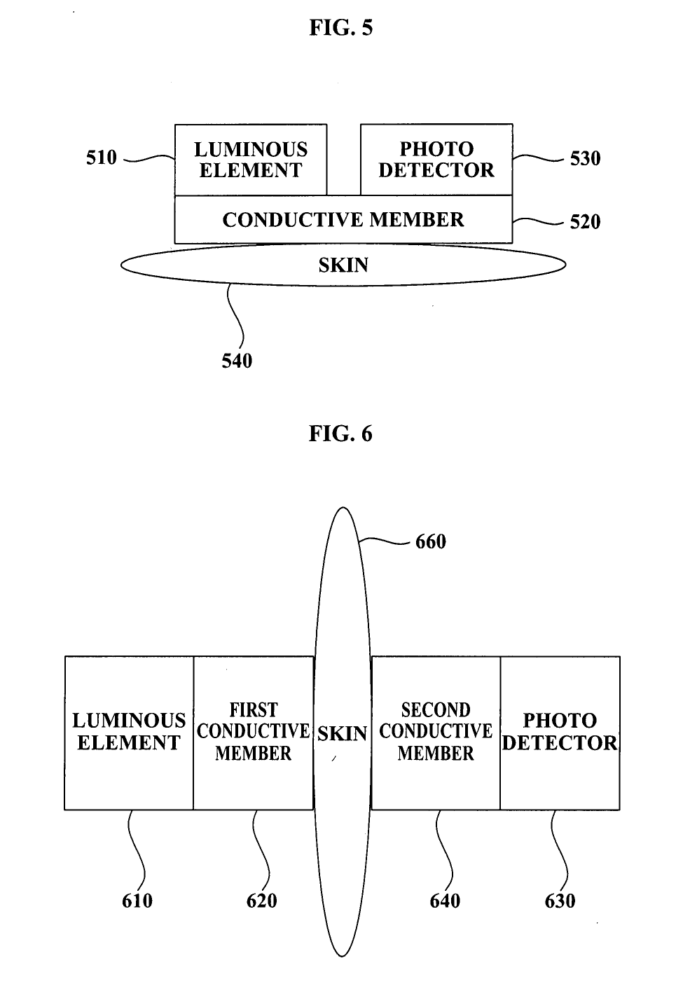

FIG. 5 illustrates a structure of a reflective PPG sensor according to still another embodiment of the present invention

The reflective PPG sensor according to the present embodiment may include a luminous element 510 and a photo detector 530 in parallel on one surface of the skin of a user 540.

Also, the reflective PPG sensor may include a conductive member 520 on both the luminous element 510 and the photo detector 530. Specifically, one surface of the conductive member 520 may contact with the skin of the user 540, and the other surface of the conductive member 520, not contacting with the skin of the user 540, may be provided with the luminous element 510 and the photo detector 530.

The conductive member 520 may include ITO glass. In this instance, the ITO glass may readily transmit light and also include conductivity. Accordingly, the ITO glass may enable the light emitted from the luminous element 510 to be sufficiently transmitted towards the skin of the user 540. Also, the ITO glass may enable the photo detector 530 to sufficiently detect the light emitted from the luminous element 510 towards the skin of the user 540.

Also, the conductive member 520 contacts with the skin of the user 540, and thereby makes the body of the user function as a ground. In this instance, the body of the user generally contacts with the earth, and thus results in the earth functioning as the ground due to the conductive member 520.

As described above, when a PPG signal of a user is measured via a PPG sensor according to the present invention, the body of the user contacts the earth so that the earth functions as a ground, instead of utilizing a ground consisting of a battery of a portable biosignal measurement device including the PPG sensor. Accordingly, a more stable earth ground may be provided, instead of a battery ground which is set to a comparatively greater value. Also, a PPG signal may be more accurately and stably detected even from the earlobe, and the like, in which the PPG signal is generally weak.

The photo detector 530 detects the light from the skin of the user 540. Specifically, the photo detector 530 detects the light which is emitted from the luminous element 510 towards the skin of the user 540 through the conductive member 520, and thereby is scattered or reflected from the skin of the user 540 through the conductive member 520.

In this instance, the conductive member 520 may include ITO glass as described above. Also, the conductive member 520 may be formed of various types of materials which can readily transmit the light and may also include conductivity.

The luminous element 510 may include an LED. Also, in addition to the LED, to measure a PPG signal, the luminous element 510 may include any type of material which is widely utilized in the art to emit the light towards the skin of the user 540.

FIG. 6 illustrates a structure of a transmitting PPG sensor according to still another embodiment of the present invention.

The transmitting PPG sensor according to the present embodiment may include a luminous element 610 on one surface of the skin of a user 660 and a photo detector 630 on the other surface of the skin of the user 660. Specifically, the transmitting PPG sensor may be formed in a type of an earring which is attachable onto the earlobe of the user.

Also, the transmitting PPG sensor may include a first conductive member 620 on the luminous element 610 and a second conductive member 640 on the photo detector 630.

Specifically, one surface of the first conductive member 620 may contact with the skin of the user 660, and the other surface of the first conductive member 620 may be provided with the luminous element 610. Also, one surface of the second conductive member 640 may contact with the skin of the user 660, and the other surface of the second conductive member 640, not contacting with the skin of the user 660, may be provided with the photo detector 630.

The first conductive member 620 and the second conductive member 640 may include ITO glass. In this instance, the ITO glass may readily transmit light and may also include conductivity. Accordingly, the ITO glass may enable the light emitted from the luminous element 610 to be sufficiently transmitted towards the skin of the user 660. Also, the ITO glass may enable the photo detector 630 to sufficiently detect the light emitted from the luminous element 610 towards the skin of the user 660.

Also, the first conductive member 620 and the second conductive member 640 contact with the skin of the user 660, and thereby make the body of the user function as a ground. In this instance, the body of the user generally contacts with the earth, and thus results in the earth functioning as the ground due to the first conductive member 620 and the second conductive member 640.

As described above, when a PPG signal of a user is measured via a PPG sensor according to the present invention, the body of the user contacts the earth so that the earth functions as a ground, instead of utilizing a ground consisting of a battery of a portable biosignal measurement device including the PPG sensor. Accordingly, a more stable earth ground may be provided, instead of a battery ground which is set to a comparatively greater value. Also, a PPG signal may be more accurately and stably detected even from the earlobe, and the like, in which the PPG signal is generally weak.

The photo detector 630 detects the light from the skin of the user 660. Specifically, the photo detector 630 detects the light which is emitted from the luminous element 610 towards the skin of the user 660 through the first conductive member 620, and thereby is scattered or reflected from the skin of the user 660 through the second conductive member 640.

In this instance, the first conductive member 620 and the second conductive member 640 may include ITO glass as described above. Also, the first conductive member 620 and the second conductive member 640 may be formed of various types of materials which can readily transmit the light and may also include conductivity.

The luminous element 610 may include an LED. Also, in addition to the LED, to measure a PPG signal, the luminous element 610 may include any type of material which is widely utilized in the art to emit the light towards the skin of the user 660.

According to the above-described embodiments of the present invention, a PPG sensor includes a conductive member, such as indium-tin oxide (ITO) glass, and the like, on one surface of an optical sensor contacting with the skin of a user, and thereby induces a more stable ground through the contacted skin of the user than a ground consisting of a battery of a portable biosignal measurement device including the PPG sensor.

Also, according to various embodiments of the present invention, there is provided a PPG sensor which reduces system noise of a measurement device through a stable ground using the skin of a user, not a battery ground, and thereby can more accurately and stably detect a PPG signal, even from the earlobe, and the like, corresponding to a weak signal source.

Although a few embodiments of the present invention have been shown and described, the present invention is not limited to the described embodiments. Instead, it would be appreciated by those skilled in the art that changes may be made to these embodiments without departing from the principles and spirit of the invention, the scope of which is defined by the claims and their equivalents.

What is claimed is:

1. A photoplethysmography (PPG) sensor, comprising:
a conductive member contacting with the skin of a user;
a luminous element disposed on the conductive member to emit light towards the skin of the user through the conductive member; and
a photo detector detecting the light emitted towards the skin of the user.

2. The PPG sensor of claim 1, wherein the conductive member includes indium-tin oxide (ITO) glass.

3. The PPG sensor of claim 1, wherein the luminous element includes a light emitting diode (LED).

4. The PPG sensor of claim 1, wherein the photo detector is disposed on the conductive member.

5. A photoplethysmography (PPG) sensor, comprising:
a conductive member contacting with the skin of a user;
a luminous element emitting light towards the skin of the user; and
a photo detector disposed on the conductive member to detect the light emitted towards the skin of the user through the conductive member.

6. The PPG sensor of claim 5, wherein the conductive member includes ITO glass.

7. The PPG sensor of claim 5, wherein the luminous element includes an LED.

8. The PPG sensor of claim 5, wherein the luminous element is disposed on the conductive member.

9. A photoplethysmography (PPG) sensor, comprising:
a conductive member contacting with the skin of a user;
a luminous element disposed on the conductive member to emit light towards the skin of the user through the conductive member; and
a photo detector disposed on the conductive member to detect the light emitted towards the skin of the user through the conductive member.

10. The PPG sensor of claim 9, wherein the conductive member includes ITO glass.

11. The PPG sensor of claim 9, wherein the luminous element includes an LED.

12. A photoplethysmography (PPG) sensor, comprising:
a luminous element emitting light towards the skin of a user;
a first conductive member disposed on one surface of the luminous element to contact with the skin of the user;
a photo detector detecting the light emitted towards the skin of the user; and
a second conductive member disposed on one surface of the photo detector to contact with the skin of the user.

13. The PPG sensor of claim 12, wherein the conductive member includes ITO glass.

14. The PPG sensor of claim 12, wherein the luminous element includes an LED.
